(19) United States
(12) Patent Application Publication
(10) Pub. No.: US 2026/0325025 A1
(43) Pub. Date: Oct. 22, 2026

(54) INTRAPARTUM MEASUREMENT NEAR-INFRARED SPECTROSCOPY DEVICE

(71) Applicants: INSTITUT NATIONAL DE LA SANTE ET DE LA RECHERCHE MEDICALE, PARIS (FR); UNIVERSITE DE PICARDIE JULES VERNE, AMIENS CEDEX 1 (FR); Centre Hospitalier Régional Universitaire d'Amiens, AMIENS CEDEX (FR); Commissariat à l'énergie atomique et aux énergies alternatives, PARIS (FR); SEENEL IMAGING, AMIENS (FR)

(72) Inventors: Fabrice WALLOIS, AMIENS CEDEX 1 (FR); Thomas FONTAINE, AMIENS (FR); Moisés Alberto DOMINGUEZ SANCHEZ, AMIENS (FR); Jeremy LARROUQUERE, AMIENS (FR); Zixi LI, AMIENS CEDEX 1 (FR); Anne PLANAT-CHRETIEN, GRENOBLE (FR); Mahdi MAHMOUD ZADEH, AMIENS CEDEX 1 (FR); Michael BERGER, CLAIX (FR)

(21) Appl. No.: 18/997,132

(22) PCT Filed: Jul. 21, 2023
(86) PCT No.: PCT/EP2023/070327

§ 371 Date: Jan. 20, 2025

(30) Foreign Application Priority Data
Jul. 22, 2022 (EP) 22306101.1

(51) Int. Cl. A61B 5/1464; A61B 5/1455; A61B 5/1459; A61B 5/386

(52) U.S. Cl. CPC A61B 5/1464; A61B 5/14552; A61B 5/14553; A61B 5/1459; A61B 5/386

(57) ABSTRACT

The present invention relates to the monitoring of the labor period. The gold standard is currently the monitoring of fetal heart rate (FHR). However, the FHR monitoring is not satisfying since it leads to low false-negative and high false-positive rates. That's why the inventors worked on a more accurate device. Thus, the present invention proposes a device adapted to achieve a smart NIRS spectroscopy which enables to accurately and rapidly recognize oxygen delivery and tissue hypoxia in the critically labor situation.

### TECHNICAL FIELD OF THE INVENTION

The present invention concerns an intrapartum measurement near-infrared spectroscopy device. The present invention also relates to an intrapartum measurement device comprising such spectroscopy device as well as a delivery monitoring system also comprising the spectroscopy device.

### BACKGROUND OF THE INVENTION

During the labor period, the infant makes the critical transition from its dependence on maternal and placental support, oxidative, nutritional, and endocrinological, and establishes independent life. The difficulty of this transition is indicated by mortality risks that are higher than any occurring until old age and by risks for damage to organ systems, most notably the brain that can be lifelong. During the labor period, the fetus has risk of severe hypoxia and metabolic acidosis. Oxygen is the most vital substrate necessary for mitochondrial respiration and is not stored in the body. Interruption of oxygen supply to cells can result in irreversible brain damage. Cerebral palsy due to intrapartum anoxia is one of the most deleterious consequences of labor.

The monitoring fetal heart rate (FHR) is currently considered as the gold standard for evaluating fetal status.

However, FHR monitoring has shown to have low false-negative and high false-positive rates.

### SUMMARY OF THE INVENTION

There is a need for developing systems enabling to accurately and rapidly recognize oxygen delivery and tissue hypoxia in the critically labor situation.

To this end, the specification describes an intrapartum near-infrared spectroscopy device adapted to carry out a near-infrared spectroscopy on the fetus's brain, the intrapartum near-infrared spectroscopy device comprising a near-infrared spectroscopy sensor and a processing circuitry. The near-infrared spectroscopy sensor is a fibered component comprising a light source and at least two light detectors, the light detectors being arranged to collect the light emitted by the fetus's brain at two distinct locations. The light emitted by the fetus's brain comprises a contribution from the surface of the skin of the fetus and a contribution from the brain. The processing circuitry is adapted to receive the signals coming from the light detectors and to process the signals of the light detectors to filter the contribution of the surface by comparing the signals coming from the light detectors and applying a diffusion model.

Such removing of the systemic component is different from the filters applied in the prior art documents, such as documents WO 2021/195299 A1, US 2004/116789 A1, U.S. Pat. No. 6,175,753 B1 and US 2016/089067 A1.

Such filters applied in the prior art documents are only trying to improving the quality of the signal, and notably parasite signal, whereas the proposed filter is adapted to select the useful part of the signal.

This part is useful with regards to the considered application of intrapartum measurement, and more precisely controlling the fetus's health.

According to further aspects of the intrapartum near-infrared spectroscopy device, which are advantageous but not compulsory, the intrapartum near-infrared spectroscopy device might incorporate one or several of the following features, taken in any technically admissible combination:
the light source comprises several excitation sources combined by couplers.
for each light detector, a central wavelength is defined, each central wavelength being comprised between 600 nanometers and 1100 nanometers.
for each excitation source, a central wavelength is defined, each central wavelength being comprised between 620 nanometers and 970 nanometers.
for each excitation source, a central wavelength is defined, each central wavelength being comprised between 600 nanometers and 1100 nanometers.
each central wavelength is comprised between 700 nanometers and 900 nanometers.
the number of excitation sources is superior to 3.
each light detector is provided with a fiber.
at least one fiber is a PMMA multimode fiber.
at least one fiber is a series of a PMMA multimode fiber and a SiO2 multimode fiber.
at least one fiber is provided with a prism.
the near-infrared spectroscopy sensor comprises more than three light detectors, notably four light detectors.
the locations are aligned.
the distance between one location and the nearest location is comprised between 2 millimeters and 15 millimeters.

The specification also describes an intrapartum measurement device comprising an intrapartum near-infrared spectroscopy device as previously described.

The intrapartum measurement device may further comprise a electroencephalography device.

The specification also concerns a delivery monitoring system comprising an intrapartum measurement device as previously described.

The specification also proposes an intrapartum measurement probe adapted to measure signals of the fetus's brain, the intrapartum measurement probe comprising a housing and a mechanical support. The housing comprises an electroencephalography sensor adapted to measure electrical signals of the fetus's brain and a near-infrared spectroscopy sensor adapted to measure fetal tissue oximetry. The mechanical support is adapted to fix the housing to the scalp or the head of the fetus.

According to further aspects of the intrapartum measurement probe, which are advantageous but not compulsory, the intrapartum measurement probe might incorporate one or several of the following features, taken in any technically admissible combination:
the electroencephalography sensor comprises two Ag/AgCl electrodes.
each electrode is used as an electromyography sensor and/or an impedance sensor.
the housing is made of two parts, each part having the form of a cup.
the mechanical support comprises a connecting structure linking the two parts and, for each part, respectively a control rod and a cable.
the mechanical support comprises a holding element adapted to hold the housing, the holding element having preferably a cup form in transverse section.
the mechanical support further comprises a protecting element adapted to protect the housing.
the intrapartum measurement probe further comprises a pressure sensor.

The specification also relates to an intrapartum measurement device comprising an intrapartum measurement probe as previously described, a first processing unit adapted to process the signals coming from the electroencephalography sensor, and a second processing unit adapted to process the signals coming from the near-infrared spectroscopy sensor.

Such probe is very original for several reasons.

The probe proposes a combination of two sensors, which have never been associated on the same probe in the specific context of intrapartum measurement.

Other associations of probe are generally used in this context, such as optical sensor and EEG sensor, as notably illustrated by document U.S. Pat. No. 5,109,849 A.

The context of intrapartum measurement is very specific in so far as the allocated space is very reduced, notably resulting in cross-talk and heating issues.

In particular, the transposition of a technique from an adult to a foetus is generally not desirable, since it is very complicated to solve these cross-talk and heating issues.

Furthermore, the position of the housing is specific.

Generally, the sensors are provided on the uterine wall so as to ensure a reproducible measurement.

On the contrary, the probe is here maintained on the scalp of the fetus, thereby enabling correct measurements.

Such probe enables to accurately and rapidly recognize oxygen delivery and tissue hypoxia in the critically labor situation.

According to further aspects of the intrapartum measurement device, which are advantageous but not compulsory, the intrapartum measurement device might incorporate one or several of the following features, taken in any technically admissible combination:
the first processing unit and the second processing unit operate in an asynchronous manner.
the first processing unit comprises a filtering unit adapted to filter the movement artifacts generated in the signals coming from the electroencephalography sensor.

The specification also describes a delivery monitoring system comprising an intrapartum measurement device as previously described.

According to further aspects of the delivery monitoring system, which are advantageous but not compulsory, the delivery monitoring system might incorporate one or several of the following features, taken in any technically admissible combination:
the delivery monitoring system further comprises measurement devices adapted to measure physical values relative to the mother.
the delivery monitoring system further comprises measurement devices adapted to measure physical values relative to the fetus, the measurement devices being positioned outside the uterus.

### BRIEF DESCRIPTION OF THE DRAWINGS

The invention will be better understood on the basis of the following description which is given in correspondence with the annexed figures and as an illustrative example, without restricting the object of the invention. In the annexed figures:

FIG. 1 is a schematic view of a delivery monitoring system comprising a intrapartum measurement device, which comprises an intrapartum measurement probe and a processing circuit,

FIG. 2 is a schematic view of the intrapartum measurement probe of FIG. 1 in position,

FIG. 3 is a perspective view of an electrode of the electroencephalography sensor of the intrapartum measurement probe,

FIG. 4 is a schematic view of a near-infrared spectroscopy device of the intrapartum measurement device of FIG. 1,

FIG. 5 is a perspective view of the intrapartum measurement probe including a mechanical support,

FIG. 6 is a view of a first part of the mechanical support,

FIG. 7 is a view in transverse section of the first part according to the axis VI-VI,

FIG. 8 is a view of a second part of the mechanical support equipped with a housing,

FIG. 9 is an exploded view of the elements represented on FIG. 8,

FIG. 10 is a view of another example of mechanical support, and

FIG. 11 is another view of the mechanical support of FIG. 10.

### DETAILED DESCRIPTION OF SOME EMBODIMENTS

### Description of the Components of the Delivery Monitoring System

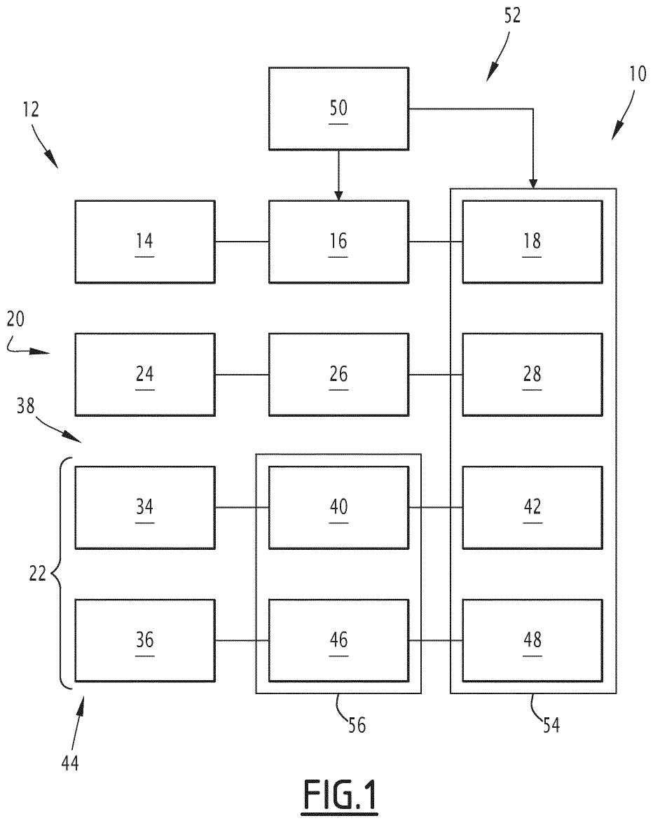

A delivery monitoring system 10 is illustrated schematically on FIG. 1.

The delivery monitoring system 10 is adapted to monitor several physical values of the mother and of the fetus in a delivery room.

The delivery monitoring system 10 may be construed as a set of measurement devices.

In the present case, the focus is on a specific part of the delivery monitoring system 10, which is the intrapartum measurement device 22.

So as to better understand the environment of such intrapartum measurement device 22, it seems important to describe an environment in which the intrapartum measurement device 22 may be used. This will notably enable to distinguish the intrapartum measurement device 22 from other measurement devices that can be found in a delivery room.

The environment is thus described hereinafter as a specific example, bearing in mind that such example is not limitative.

Each measurement device comprises a sensor, a processing circuit and an output unit.

The measurement devices measure physical values either of the mother either of the fetus.

The measurement devices providing with physical values relative to the mother are named mother measurement devices 12.

For instance, the mother measurement devices 12 comprise a heart rate detector, an abdominal electrode or any measuring device that can be relevant in the context.

Although in general all these mother measurement devices 12 are separate elements, they are represented as a unique measurement device in FIG. 1.

Therefore, the delivery monitoring system 10 comprises a mother sensor block 14, a mother processing circuit block 16 and a mother output unit block 18.

Concerning the fetus, the physical values are either measured from outside or intrapartum.

For clarity, the measurement devices providing with physical values relative to the fetus from outside are named outside fetus measurement devices 20 and the measurement device providing with physical values relative to the fetus intrapartum is named intrapartum measurement device 22.

For instance, the outside fetus measurement devices 20 comprise a heart rate detector or any measuring device that can be relevant in the context.

Similarly to the mother measurement devices 12, although in general all these outside fetus measurement devices 20 are separate elements, they are represented as a unique measurement device in FIG. 1.

Therefore, the delivery monitoring system 10 comprises an outside fetus sensor block 24, an outside fetus processing circuit block 26 and an outside fetus output unit block 28.

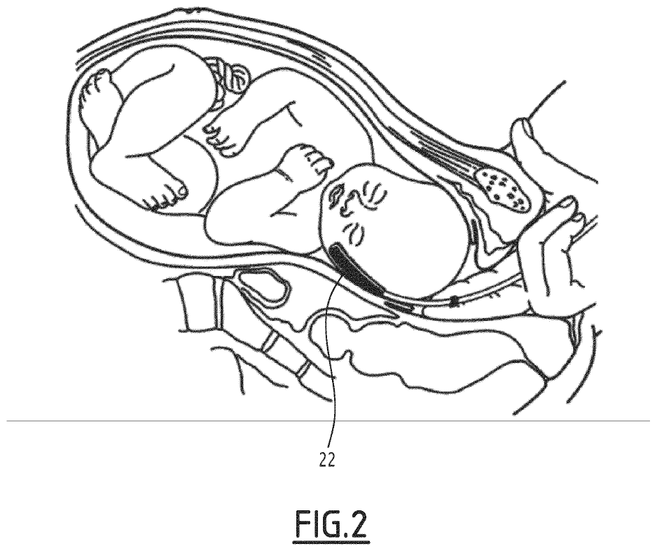

The intrapartum measurement device 22 is illustrated in position, this is inside the uterus, on FIG. 2.

The intrapartum measurement device 22 is a device adapted to measure signals of the fetus's brain.

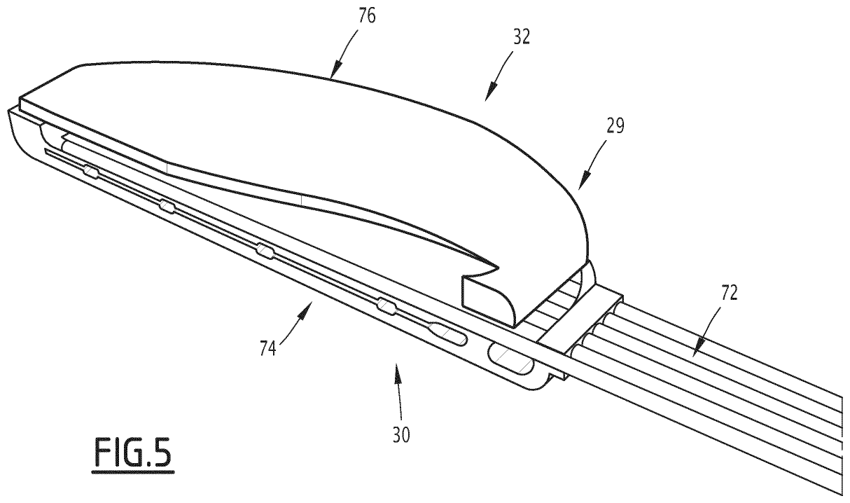

The intrapartum measurement device 22 comprises an intrapartum measurement probe 29 made of a housing 30 and a mechanical support 32, which can be seen on FIGS. 5 to 9.

The housing 30 comprises an electroencephalography sensor 34 and a near infra-red spectroscopy sensor 36.

Hereinafter, the electroencephalography sensor 34 is named EEG sensor 34, EEG being the abbreviation of electroencephalography.

Similarly, the near-infrared spectroscopy sensor 36 is named NIRS 36 sensor hereinafter. NIRS being the abbreviation of near-infrared spectroscopy.

As an initial comment, it can be noticed that the intrapartum measurement device 22 is a bimodal measurement device for the fetus's brain since it comprises two distinct measurement modalities, namely a first modality corresponding to the EEG sensor and a second modality corresponding to the NIRS sensor.

The EEG sensor 34 is part of a EEG device 38, which also comprises an EEG processing circuit 40 and a EEG output unit 42.

Similarly, the NIRS sensor 36 is part of a NIRS device 44, which also comprises a NIRS processing circuit 46 and a NIRS output unit 48.

In the present case of FIG. 1, each processing circuit is controlled by a controller 50, which is part of a computer 52.

This computer 52 is also provided with a display device 54, which is a screen. Such display device 54 is shared by the different measurement devices 12, 20 and 22.

This means that the mother output unit block 18, the outside fetus output unit block 28, the EEG output unit 42 and the NIRS output unit 48 are realized as a unique unit, which is the display device 54.

As a consequence, according to the embodiments, the display device 54 may display one or several of the following physical values: the value of the fetal EEG, the values of cerebral hemodynamics, the value or the fetal heart rate, the value of the maternal heart rate and the value of the contractions.

Furthermore, the EEG processing circuit 40 and the NIRS processing circuit 46 are realized on the same PCB (printed cardboard) so that they share similar components. The set of the EEG processing circuit 40 and the NIRS processing circuit 46 forms a probe processing circuit 56.

However, according to another embodiment the EEG processing circuit 40 and the NIRS processing circuit 46 are separate and are realized on different PCBs.

### Description of an Example of EEG Sensor 34

The EEG sensor 34 is adapted to measure electrical signals of the fetus's brain.

The electrical signals of the fetus's brain correspond to the neuronal activity of the fetus's brain.

Therefore, the EEG sensor 34 is a sensor adapted to acquire the neuronal activity of the fetus's brain.

The EEG sensor 34 comprises two electrodes 58, each of these electrodes being an Ag/AgCl electrode.

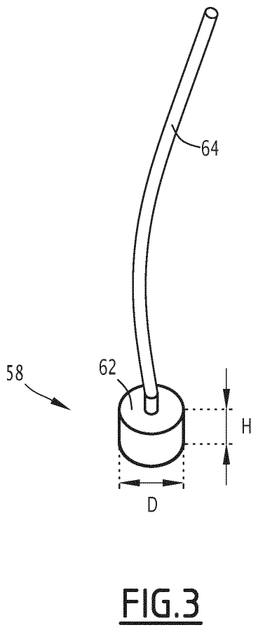

One electrode 58 is represented on FIG. 3.

Each electrode 58 has a cylindrical shape 62.

The basis of the cylindrical shape 62 is a circle whose diameter D is comprised between 1 mm and 8 mm.

The height H of the cylindrical shape 62 is preferably inferior to the diameter of the circle.

Each electrode 58 is connected to an embedded wire 64, notably a silver wire. This renders connected to any cable or other conductive media by soldering or crimping them to the embedded wire 64.

These sintered Ag/AgCl electrodes 58 are very adapted to this specific case of intrapartum measurement. They notably enable to extract more information from a small fetus brain, allowing more degrees of freedom, since they benefit from the low noise level it generates during biological signals recording.

In addition, the electrodes 58 can be used to record uterus contraction by being used as an electromyography (EMG) sensor. In such configuration, the EEG is on the side of the head of the fetus while the EMG is on the side of the uterine wall.

Alternatively, the record uterus contraction can be carried out by a pressure sensor.

The electrodes 58 can also be used to carry out an impedance measurement to measure the probe attachment. The impedance measured here is the impedance of the skin with which the probe is attached.

### Description of an Example of NIRS Device 44

The NIRS sensor 36 is adapted to measure fetal tissue oximetry.

Given the position of the intrapartum measurement device 22, this means that the NIRS sensor 36 is adapted to measure the fetal cerebral oximetry.

In the present case, the NIRS sensor 36 is a fibered component.

A fiber component enables to meet security constraints on the one hand, and to avoid cross-talk between modalities on the other hand.

It can be noted here that using a fibered component is a way of fulfilling such constraints but other means could be considered such as embedded led and detector into the probe or electronic connection with the main board or communication through wireless connection.

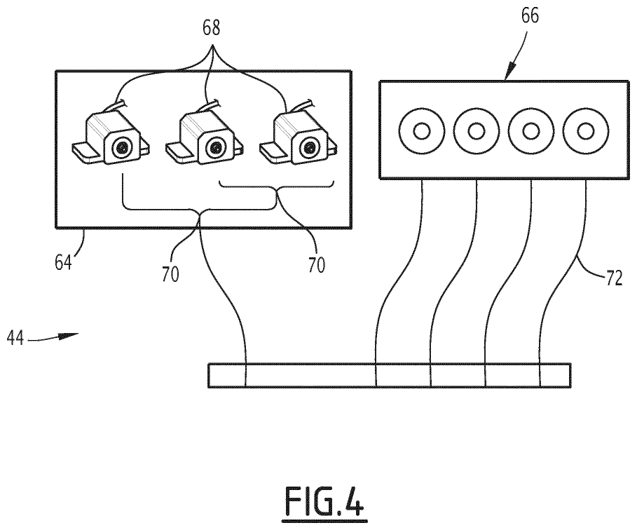

More precisely, the NIRS sensor 36 comprises a light source 64 and at least two light detectors 66, four in the case of the example of FIG. 4. With the Applicant's tries, four appears to be a good trade-off between precision and low volume of the NIRS sensor.

The light source 64 is formed by the excitation coming from several excitation sources 68.

Each excitation source 68 is a fibered LED.

For each of these excitation sources 68, a central wavelength can be defined.

The NIRS sensor 36 is adapted to send a minimum of 2 central wavelengths in an optical windows in NIRS corresponding to the biological tissue, namely between 600 nanometers (nm) and 1100 nm.

This means that for, each light detector, a central wavelength is defined, each central wavelength being comprised between 600 nanometers and 1100 nanometers.

In the present case, the number of excitation sources 68 is equal to three and each of the central wavelengths are comprised between 620 nm and 970 nm, preferably between 720 nm and 900 nm.

The excitation sources 68 are combined by optical couplers 70. Other means can nevertheless be considered.

The light detectors 66 are arranged to collect the light emitted by the fetus's brain in response to the light emitted by the light source 64 at four different locations.

For this, each light detector 64 is provided with a respective fiber 72.

According to a first example, the fiber 72 is a PMMA multimode fiber.

According to another example, so as to limit the loss, the fiber 72 is a set of a PMMA multimode fiber followed by a SiO₂ multimode fiber.

According to still another example, a set of fiber in SiO₂ with a prism is used.

The collection of the light emitted by the fetus's brain at different locations enables to separate two contributions, one coming from systemic contribution (from the surface of the skin of the fetus) and the other one coming from the brain (deep contribution).

In that sense, the NIRS device 44 is a functional NIRS device since it is adapted to provide with in-depth quantitative estimation of oxygenation variations by separating systemic variations (surface area) from cerebral variations. The NIRS device can be used for separating the layers contribution, in order to access the oxygen information at the ‘brain level’.

For this, the NIRS processing circuitry 46 is adapted to receive the signals measured by each light detector 66.

The NIRS processing circuitry 46 is further adapted to process the signals of the light detectors to filter the contribution of the surface by comparing the signals coming from the light detectors 66 and applying a diffusion model.

As a specific example, the diffusion model could be derived from Beer-Lambert law or by using a spatially resolved spectroscopy technique, often named SRS technique.

SRS technique is notably used in NIRS to obtain tissue oxygenation index (TOI). The minimal contamination from superficial layers is obtained by measuring the gradient of light-intensity attenuation at multiple wavelengths. For this, a set of closely placed detectors is used at nearly the same source-detector distance, large enough to probe the cerebral tissue

The filtering part corresponds to exploiting the property according to which it is generally considered that the tissue depth imaged is equal to half the distance between the emitter and the detector. This means that for short distances between the emitter and the detector (short channel) only the surface contribution is obtained and for long distances between an emitter and the corresponding detector (long channel), surface and cerebral tissue are imaged. By smartly filtering the long channel images by one or several short channel images, the systemic contribution is eliminated.

So as to increase such filtering approach of the systemic contribution, according to one embodiment, the four locations are aligned.

The same effect is obtained for three aligned locations.

In variant, the locations are arranged along other forms, such as circle or a square

It is also preferred that the distance between one location and the nearest location be comprised between 2 mm and 15 mm.

### Description of an Example of Mechanical Support 32

An example of mechanical support 32 is illustrated by FIGS. 5 to 9.

The mechanical support 32 comprises two parts: a holding element 74 and a protecting element 76.

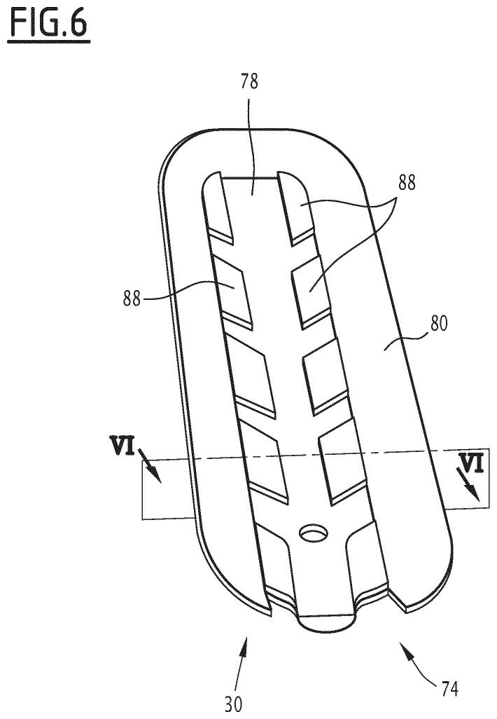

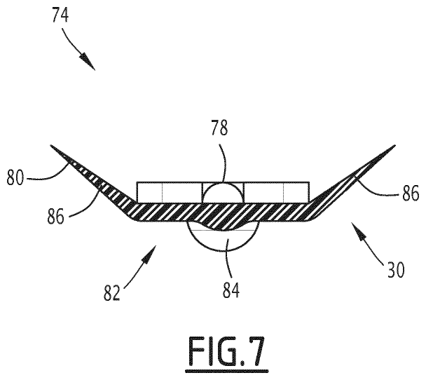

The holding element 74 can be viewed in more details on FIGS. 6 and 7.

The holding element 74 is adapted both to hold the housing 30 and ensure that the intrapartum measurement probe 29 be held in place by pressure, wedged between the head of the fetus and the uterine wall.

As a specific example, the holding element 74 is made in a gecko adhesive material.

The holding element 74 comprises a central portion 78 and a peripheral portion 80 surrounding the central portion.

Seen from above, the holding element 74 has a general form of a rectangle while seen from a side, the transversal section (along the axis VI-VI on FIG. 6) has a form of a cup 82 with a bottom 84 and two sides 86 extending from the bottom towards the exterior.

The bottom 84 of the cup 82 corresponds to the central portion 78 while the sides 86 of the cup 82 correspond to the peripheral portion 80.

The bottom 84 and each side 86 form an angle comprised between 35° and 45°.

In the present case, the angles formed by the bottom 84 and each side 86 have the same values to simplify the manufacturing (by molding notably) of the holding element 74.

The central portion 78 is provided with fixing elements 88.

The fixing elements 88 protrude from the central portion 78 and are intended to collaborate with complementary elements present on a face 90 of the housing 30.

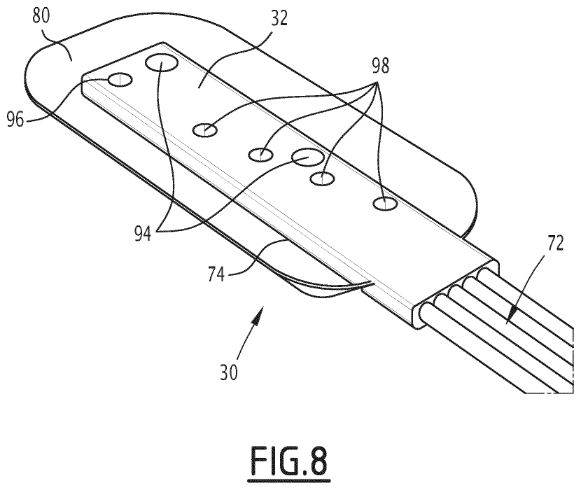

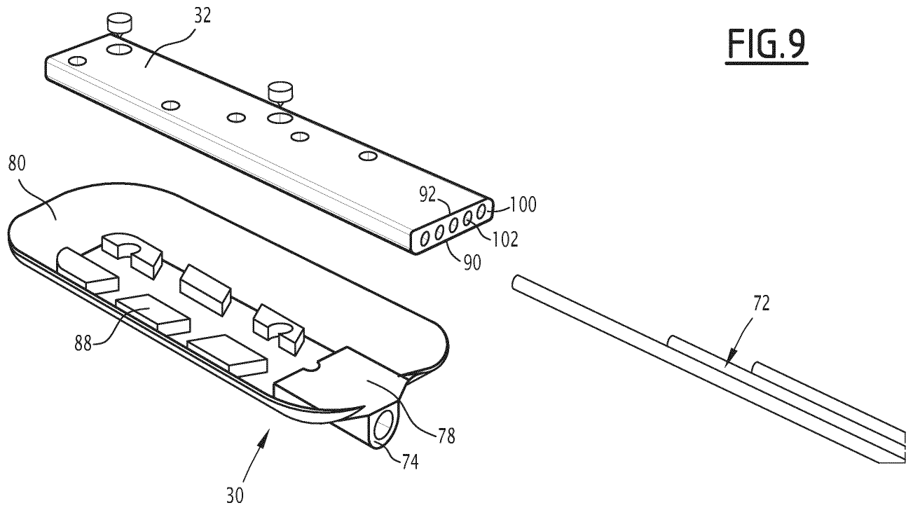

The other face 92 of the housing 30 can be seen on FIGS. 8 and 9.

The other face 92 has a rectangular shape with a length comprised between 20 mm and 40 mm and a width comprised between 4 mm and 10 mm.

The other face 92 comprises seven openings: two for the electrodes (electrode opening 94 hereinafter), one for the insertion of the fiber of the light source (light source opening 96 hereinafter) and four for the insertion of the fours fibers of the light detectors (light detector opening 98 hereinafter).

The distance between the two electrode openings 94 is comprised between 15 mm and 30 mm.

In addition, the distance between the light source opening 96 and the nearest light detector opening 98 is comprised between 2 mm and 20 mm.

One electrode opening 94 is located in front of the light source opening 96 while the other electrode opening 94 is located between two light detector openings 98, more precisely between the central light detector openings 98.

Such arrangement enables to avoid crosstalk between both modalities (EEG and NIRS).

The fibers 72 are fixed by one lateral side 100 of the housing 30, which enables to obtain a quite low voluminous housing 30. For this, the lateral side is provided with holding openings 102 through with the fibers 72 are inserted.

The protecting element 76 is adapted to protect the openings 94, 96 and 98, and more precisely the sensors 34 and 36.

### Description of Another Example of Mechanical Support 32

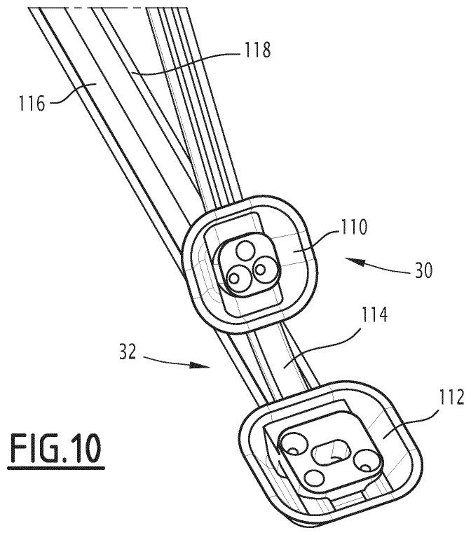

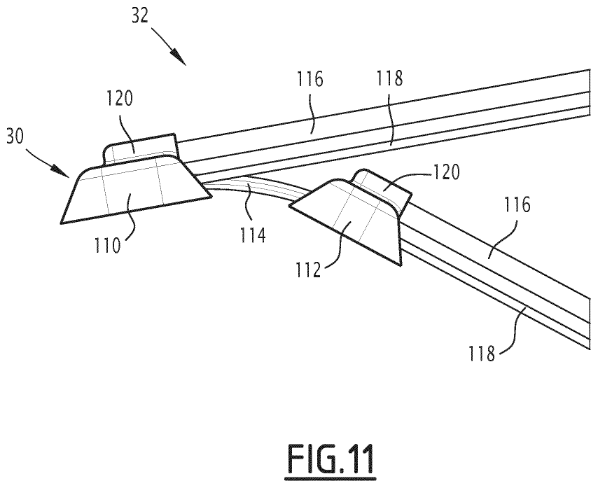

Another example of mechanical support 32 is illustrated on FIGS. 10 and 11.

The housing 30 comprises here two parts 110 and 112.

The different elements of the intrapartum measurement probe are shared between the two parts 110 and 112.

Each part 110 and 112 has the general form of a cup.

In addition, each part 110 and 112 holds on the head of the fetus by suction, so that the parts 110 and 112 correspond a suction cup.

The mechanical support 32 comprises a connecting structure 114 linking the two parts and 112 and, for each part 110 and 112, respectively a control rod 116 and a cable 118.

The connecting structure 114 is soft enough to follow the head curvature of the fetus.

As apparent from FIG. 11, each control rod 116 is linked to its respective part 110 or 112 by a receptacle 120, the receptacle being fixed to the bottom of the part 110 or 112.

The control rods 116 enable to control the angle between the two parts 110 and 112 by pushing one and/or pulling the other one.

Each cable 118 embeds one beam of optical fibers.

### Description of an Example of Processing Circuitry

The EEG processing circuit 40 and the NIRS processing circuit 46 have different sampling rates so that they work in an asynchronous manner.

This implies that the probe processing circuit 56 is provided with a synchronization unit adapted to synchronize the data coming from the EEG processing circuit and the NIRS processing circuit by using their local timestamp.

In other words, each of the EEG processing circuit 40 and the NIRS processing circuit 46 are able to timestamp an element with a timestamp derived from their own sampling rates. The synchronization unit will thus calculate the real time and synchronize the data with the same “absolute” timestamp.

### Interests of the Intrapartum Measurement Device 22

The intrapartum measurement device 22 enables to address the challenge of designing and developing a device and probe that provide reliable access for the monitoring of fetal cerebral oxygenation simultaneously to neuronal activity and heart rate while the cervix must be sufficiently dilated and the membranes ruptured.

Notably, the scalp is covered by hair, and mucous, blood etc., all of which can interfere with light transmission and photodetection. In addition, a good contact between the probe and the skin should also be obtained while pressure sores from the probes should be minimized.

For this, the present intrapartum measurement device 22 fulfills many technical constraints, among which medical constraints of maneuverability, accessibility, compatibility, technical constraints related to electrodes and EEG signal analysis, technical constraints related NIRS carrying out and NIRS signal analysis and modality mergers constraints.

Such intrapartum measurement device 22 is provided with synchronization and the real-time functionalities, which renders such device adapted to be used as an autonomous monitoring device.

Combining an EEG sensor 34 and a NIRS sensor 16 in the same intrapartum measurement device 22 provides with a simultaneous measurement bimodality ability.

Such property enables to address the function/dysfunction of both hemodynamic and neuronal activities independently but also conjointly so as to minimize misleading interpretation arising from one or the other modalities.

The intrapartum measurement device 22 also provides with a better understanding of the mechanisms involved in this situation of normal or pathological delivery.

In addition, such intrapartum measurement device 22 enables to address specifically neurovascular origin and consequences of an acute cerebral distress during delivery. Such knowledge enables to prevent neurological sequels of postpartum, which appear later in development and can be declined in the form of various brain diseases ranging from death, cerebral palsy and cognitive disorders.

The intrapartum measurement device 22 also provides with information on cerebral function/dysfunction in all situations where one or the other of the modalities would be inoperative, thus making it possible to go beyond the limits of each of the techniques that have hitherto been proposed.

The intrapartum measurement device 22 enables to accurately and rapidly recognize oxygen delivery and tissue hypoxia in the critical labor situation.

The intrapartum measurement device 22 also strengthens the bundle of arguments considering the opportunity of a C-section.

### Other Embodiments of the Intrapartum Measurement Device 22

Other embodiments of the intrapartum measurement device 22 may be considered. Some examples are given hereinafter.

As a first example, the intrapartum measurement device 22 further comprises a set of biosensors.

For instance, the intrapartum measurement device 22 also comprise a pressure sensor.

As a second example, since the EEG signals of human fetus are generally very weak, the intrapartum measurement device 22 is provided with a specific detecting module.

For instance, the detecting module comprises a filtering unit.

The filtering unit is adapted reduce the EEG signal contamination induced by movement artifacts, notably labor.

As a specific embodiment, the movement artifacts may be detected by using the pressure sensor mentioned in the first example of this paragraph.

Alternatively or in complement, the detecting module may also comprises a preamplifier with high common-mode rejection ratio and high signal-to-noise ratio.

1. An intrapartum near-infrared spectroscopy device adapted to carry out a near-infrared spectroscopy on the fetus's brain, the intrapartum near-infrared spectroscopydevice comprising:
a near-infrared spectroscopy sensor, said near-infrared spectroscopy sensor being a fibered component comprising a light source and at least two light detectors, the light detectors being arranged to collect the light emitted by the fetus's brain at two distinct locations, the light emitted by the fetus's brain comprising a contribution from the surface of the skin of the fetus and a contribution from the brain, and
a processing circuitry adapted to receive the signals coming from the light detectors and to process the signals of the light detectors to filter the contribution of the surface by comparing the signals coming from the light detectors and applying a diffusion model.

2. The intrapartum near-infrared spectroscopy device according to claim 1, wherein the light source comprises several excitation sources combined by couplers.

3. The intrapartum near-infrared spectroscopy device according to claim 1, wherein for each light detector, a central wavelength is defined, each central wavelength being comprised between 600 nanometers and 1100 nanometers.

4. The intrapartum near-infrared spectroscopy device according to claim 1, wherein for each excitation source, a central wavelength is defined, each central wavelength being comprised between 620 nanometers and 970 nanometers.

5. The intrapartum near-infrared spectroscopy device according to claim 2, wherein the number of excitation sources is superior to 3.

6. The intrapartum near-infrared spectroscopy device according to claim 1, wherein each light detector is provided with a fiber.

7. The intrapartum near-infrared spectroscopy device according to claim 6, wherein at least one fiber is a PMMA multimode fiber.

8. The intrapartum near-infrared spectroscopy device according to claim 6, wherein at least one fiber is a series of a PMMA multimode fiber and a SiO₂ multimode fiber.

9. The intrapartum near-infrared spectroscopy device according to claim 1, wherein at least one fiber is provided with a prism.

10. The intrapartum near-infrared spectroscopy device according to claim 1, wherein the near-infrared spectroscopy sensor comprises more than three light detectors.

11. The intrapartum near-infrared spectroscopy device according to claim 10, wherein the locations are aligned.

12. The intrapartum near-infrared spectroscopy device according to claim 1, wherein the distance between one location and the nearest location is comprised between 2 millimeters and 15 millimeters.

13. An intrapartum measurement device comprising an intrapartum near-infrared spectroscopy device according to claim 1.

14. The intrapartum measurement device according to claim 13, wherein the intrapartum measurement device further comprises a electroencephalography device.

15. A delivery monitoring system comprising an intrapartum measurement device according to claim 13.
